(19) United States
(12) Patent Application Publication
(10) Pub. No.: US 2024/0398706 A1
(43) Pub. Date: Dec. 5, 2024

(54) INJECTABLE FORMULATIONS OF APREPITANT

(71) Applicant: Good Health, LLC, Princeton, NJ (US)

(72) Inventor: Basavaraj Siddalingappa, Gujarat (IN)

(73) Assignee: Good Health, LLC, Princeton, NJ (US)

(21) Appl. No.: 18/806,339

(22) Filed: Aug. 15, 2024

Related U.S. Application Data
(63) Continuation-in-part of application No. 17/804,204, filed on May 26, 2022.

(30) Foreign Application Priority Data
May 31, 2021 (IN) 202141024183

(51) Int. Cl. A61K 9/107; A61K 9/00; A61K 31/5377; A61K 47/10; A61K 47/12; A61K 47/24; A61K 47/26; A61K 47/44

(52) U.S. Cl. CPC A61K 9/107; A61K 9/0019; A61K 31/5377; A61K 47/10; A61K 47/12; A61K 47/24; A61K 47/26; A61K 47/44

(57) ABSTRACT

Injectable aprepitant emulsion formulations having enhanced stability and devoid of precipitate for extended periods of time are disclosed. Methods of preparing the same and use of the same are also disclosed.

### CROSS-REFERENCE TO RELATED APPLICATIONS

This application is a continuation-in-part of U.S. patent application Ser. No. 17/804,204, filed May 26, 2022, which in turn, claims the benefit of priority from Indian patent application No. 202141024183 filed May 31, 2021, the contents of each of which are incorporated herein by reference.

### FIELD OF INVENTION

This invention relates to a stable, physiologically compatible emulsion formulations of aprepitant intended for intravenous injection.

### BACKGROUND OF THE INVENTION

Aprepitant is an antiemetic drug used to treat nausea and vomiting induced by chemotherapy. It was initially available as an oral capsule.

Aprepitant is a poorly water soluble drug and it is slowly absorbed after oral administration, with oral bioavailability of about 69%. Moreover, due to prevailing conditions such as nausea and vomiting, it is difficult for some patients to swallow a capsule and sustain it in the body. Due to vomiting, most of the drug could come out.

Intravenous administration of aprepitant is recommended for patients who cannot take oral dosage forms. Fosaprepitant dimeglumine is water soluble prodrug salt of aprepitant and is available as a lyophilized powder for intravenous injection after reconstitution and dilution.

U.S. Pat. No. 9,561,229 discloses an injectable pharmaceutical emulsion formulation of aprepitant, which contains from 0.4 to 1% W/W aprepitant, 13-16% egg lecithin, 9 to 10% of soya bean oil and the pH modifier sodium oleate. The formulations have a pH of between 7.5 to 9.0.

U.S. Pat. No. 9,808,465 teaches about emulsion formulations of Aprepitant for injection. The emulsion includes 11-15% of emulsifier, oil, an alcohol, tonicity modifier, and a pH modifier, wherein emulsifier to aprepitant ratio is between 18:1 to 22:1 and the pH is between 7.5 to 9.0.

U.S. Pat. No. 9,974,742 also discloses aprepitant emulsions for injection as in case of U.S. Pat. No. 9,808,465. It also provides a specific process to make an emulsion wherein the oil phase is mixture of alcohol, aprepitant, emulsifier and the water phase is made of water/buffer, tonicity adjusting agent and pH modifier. The oil phase and water are homogenized to get a pharmaceutical emulsion, which is further sterilized to make it suitable for injection.

U.S. Pat. No. 9,974,793 discloses an emulsion formulation of aprepitant which comprises soya bean oil, egg lecithin and water wherein the ratio of egg lecithin to aprepitant is 20:1 and the ratio of oil to aprepitant is 13:1.

U.S. Pat. No. 9,974,794 also discloses emulsion formulations of aprepitant similar to those disclosed in U.S. Pat. No. 9,561,229 but the '794 patent specifies the pH modifier as being sodium oleate.

U.S. Pat. No. 10,500,208 also discloses formulations and processes of making aprepitant emulsions as in case of U.S. Pat. No. 9,974,742, however in this case, additional homogenization cycles are specified.

U.S. Pat. No. 10,624,850 again discloses emulsions which contain aprepitant, emulsifier, oil, pH modifier, co-surfactant, tonicity agent and water.

What is not disclosed in above the patents is an emulsion formulation of aprepitant with lower amounts of emulsifier which are nonetheless stable for longer time at room temperature. The present invention addresses this shortcoming.

### SUMMARY OF THE INVENTION

This invention relates to injectable emulsion formulations of aprepitant suitable for inclusion in glass vials. In some aspects, the injectable aprepitant emulsion, comprise:
a) from about 0.25 to about 1.5% W/W aprepitant;
b) from about 8 to about 10.8% W/W of a phosphatidylcholine emulsifier;
c) from about 1.25 to about 2.8% W/W of a cosolvent comprising ethanol;
d) from about 7 to about 10.5% W/W of an oil;
e) from about 0.2 to about 1.0% W/W oleic acid or an oleic acid salt;
f) from about 0.25 to about 0.5% W/W sodium benzoate;
g) from about 4 to about 6% W/W of a tonicity agent selected from the group consisting of propylene glycol, sucrose and mixtures thereof;
h) from about 65 to about 80% W/W of water; and
i) from about 0.05 to about 1.5% W/W of a base.

The aprepitant emulsions have (i) an initial pH of greater than 9.5, (ii) preferably, a mean globule size (Z-average) of between about 60 and about 160 nm, more preferably between about 80-100 nm or between 90-100 nm, and preferably have total impurities of less than 0.1% after 6 months at 25° at 60% relative humidity.

In alternative embodiments, the injectable aprepitant emulsions are surfactant free.

This invention also describes methods of treatment of patients in need of treatment with compositions of the invention. Such methods include administering a drug-containing composition as described herein to a patient in need of such drug, preferably by administering the composition as intravenous push or IV infusion with further dilution of composition of the formulation in the infusion bag prior to administration.

The invention also relates to methods of making the aprepitant emulsion compositions described herein and methods of packing compositions of this invention into suitable vials.

### DETAILED DESCRIPTION OF THE INVENTION

The invention relates to stable, liquid pharmaceutical compositions comprising aprepitant, at least one lipid excipient, an oil, a surfactant, a co-surfactant, organic salts, solvent and tonicity agent. Within this broad aspect of the invention, some inventive compositions include injectable aprepitant emulsion formulations which include aprepitant, an oil, a phosphatidylcholine emulsifier, a surfactant, a cosolvent, water, a pH modifier or pH adjusting agent, and optionally, a salt of an organic acid. The aprepitant containing formulations are oil in water emulsions. The phosphatidylcholine emulsifier included in the formulations is present in the emulsion at concentration of <11.0% W/W and the initial pH of the emulsion formulation is ≥9.5 or ≥9.9. In other aspects, the initial pH is between about 9.0 and about 10.8 and can be preferably between about 10.0-about 10.5. This initial pH is the value determined just after preparation of product, i.e. within minutes of completion of the batch, up to about 2-3 hours. The inventive emulsion formulations are suitable for IV (intravenous) administration to patients in need of treatment or prophylaxis of nausea or vomiting or any other condition for which aprepitant is known to be useful.

In accordance with this aspect, the methods include administering an effective amount of the aprepitant emulsion compositions described herein to patient, i.e. human or other mammal in need of such treatment. For purposes of the present invention, an “effective amount” shall be understood to mean an amount sufficient to have a desired therapeutic effect on the patient being treated, i.e. sufficient to at least temporarily prevent or diminish the symptoms of nausea and/or vomiting. Further information related to the generally accepted dosages and administration schedules can be found in the FDA-approved package insert for Cinvanti®, aprepitant, the contents of which are incorporated herein by reference.

Preferably, the aprepitant is in its native form or its pharmaceutically acceptable salt, ester, or prodrug thereof. The term “aprepitant” includes aprepitant or a pharmaceutically acceptable salt, ester, or prodrug thereof. The aprepitant may alternatively be in the form of a complex. The aprepitant may be present in the compositions of the invention in any amount, such as an amount ranging from about 0.25% W/W to about 1.5% W/W, preferably 0.65 to about 0.75% W/W.

The emulsifier present in compositions of this invention are lecithins and are more preferably predominantly phosphatidylcholines which may be derived from natural, semi-synthetic or synthetic source but are in most aspects of the invention obtained from natural sources. Suitable phosphatidylcholines used in the inventive compositions are either from soya or egg lecithin such as egg phosphatidylcholine which is preferred in some aspects of the invention. In addition, the phosphatidylcholines (PC's) included in the compositions of this invention are pharmaceutically acceptable PC's and purified forms of lecithin, with phosphatidylcholine content >80%, preferably >85% W/W, more preferably >90% W/W, most preferably >92% W/W.

The amount of emulsifier, i.e., PC, present in the formulation is <11% W/W. In some aspects of the invention, the amount of the phosphatidylcholine emulsifier in the formulation is from about 8 to about 10.8% W/W of the formulation. In further aspects, the amount of PC in the emulsion is >about 8% W/W or >about 9% W/W, or about 10% W/W, but still <11% W/W. Moreover, the sum of the emulsifier and any optional surfactant, if included, is preferably below 11% W/W.

The injectable aprepitant emulsion formulations of the invention also include an oil which is preferably a vegetable oil such as soya bean oil in amount of from about 7 to about 10.5% W/W of the formulation. In alternative aspects, the amount of vegetable oil in the composition is at least about 8% W/W or at least about 9% W/W and, in some alternative embodiments, from about 9.5 to about 10.5% W/W.

In some preferred embodiments, the aprepitant emulsions are surfactant free. As will be seen from the examples below, it has been surprisingly found that formulations which are surfactant free have dramatically improved long-term stability.

Alternatively, some compositions of this invention can contain a surfactant belonging to synthetic or semi-synthetic class such as an ester of a fatty acids or ester of vitamin E or a ester of phospholipid such as either derivative of vitamin E or phospholipid like Vitamin TPGS or Pegylated phospholipids such as 1,2-distearoyl-sn-glycero-3-phosphoethanolamine-N-[amino (polyethylene glycol)-2000] Na, DSPE-PEG2000, Na or Vitamin E TPGS or Polysorbate 80.

In these alternative embodiments, the amount of surfactant in theses alternative formulations can be from about 0.1 to about 0.8% W/W of the formulation in some aspects. In others, the amount of surfactant in emulsion formulation ranges from 0.1 to 0.6% W/W. Some embodiments of this aspect of the invention include amounts of surfactant in the emulsion of at least about 0.2%, or at least about 0.3% W/W or 0.4% W/W or 0.5% W/W, while in still further embodiments it is from 0.55 to 0.6% W/W.

The emulsion compositions of the invention will also contain a cosolvent, which can be an organic solvent such as short chain alcohol, like ethanol or benzyl alcohol. Other cosolvents useful in the formulations of the present invention include polyethylene glycol, propylene glycol or mixtures thereof, or optionally combined with ethanol. Preferably, the organic cosolvent is ethanol. The amount of the cosolvent in the formulation is from about 1.25 to about 2.8% W/W of the formulation in most embodiments. Alternatively, the amount of organic solvent in the composition can be from about 1 to about 4% W/W.

The amount of water in the injectable aprepitant emulsion formulations described herein can be from about 65 to about 80% W/W of the formulation, with amounts of about 70-76% W/W in some embodiments.

The inventive aprepitant emulsions include a pH modifier which is preferably oleic acid or an oleic acid salt. Alternatively, the pH modifier can be a salt of a fatty acid such as the sodium salts thereof, e.g., sodium oleate. In many aspects of the invention, the amount of the pH modifier is from about 0.2 to about 1% W/W of the formulation. In alternative aspects, the amount of pH modifier in the composition is from about 0.2 to about 0.8% W/W, or from about 0.4 to about 0.6% W/W. Although the oleic acid can be added to either the water or oil phase, in some preferred embodiments, the oleic acid is added to the oil phase.

After the initial pH measurement discussed above, the compositions of this embodiment have a pH value of at least 7.5. In some embodiments the PH is in the range of between 7.5-10.5 or between 8.5-10.8. This pH value is observed a short period, e.g. a few days, after formation of the emulsion, and is to be contrasted with the initial pH value which is measured substantially immediately after the formation of the emulsion. Preferably, the fatty acid content of the oleate is high, e.g. at least about 80% and the alkalinity of oleate when tested as an aqueous solution is also relatively high, e.g. at least about pH of 10.3 of 0.55% W/V solution in water.

The injectable aprepitant emulsion formulation can also include adjunct pH modifiers, tonicity agents, buffering agents, antioxidants, chelating agents and preservatives.

Tonicity agents suitable for inclusion in the aprepitant compositions of the invention include tonicity adjusting agents such as sodium chloride, glycerol, propylene glycol, dextrose, lactose, mannitol, sorbitol, sucrose. In some preferred aspects, the tonicity agent is propylene glycol, sucrose or a combination thereof.

Some formulations in accordance with the invention include the tonicity agent propylene glycol in an amount of from about 2 to about 4% W/W of the formulation. Others include the tonicity agent sucrose in amounts of from about 4 to about 6% W/W of the formulation. Sucrose is a preferred tonicity agent.

The preferred antioxidants for the compositions are sodium meta bisulfate, BHA. BHT, monothioglycerol, Vitamin E and its esters.

The inventive emulsion compositions of this invention may also contain an organic acid salt in amounts of at least about 0.25% W/W, or from about 0.25 to about 0.5% W/W. Suitable organic salts are salts of benzoic acid, such as preferably sodium benzoate.

In view of the foregoing, some suitable formulations in accordance with the invention are set forth in Table 1:

| Ingredients | Qty (Mg/mL) | | % W/W | Range (%) |
|---|---|---|---|---|
| Aprepitant | 7.22 | mg | 0.722 | 0.25-1.5 |
| Egg Phosphatidylcholine | 100 | mg | 10.0 | 8-10.8 |
| Ethanol | 16.67 | mg | 1.667 | 1.25-2.80 |
| Soyabean oil | 88.8 | mg | 8.88 | 7-10.5 |
| Oleic acid | 5.32 | mg | 0.532 | 0.2-1.0 |
| NaOH | 1.20 | mg | 0.120 | 0.05-1.5 |
| Sodium Benzoate | 4.62 | mg | 0.462 | 0.25-0.5 |
| Sucrose | 55.5 | mg | 5.55 | 4-6 |
| Milli-Q-Water | 709.35 | mg or Qs | | |
| pH Range (not initial) | | | | 7.5 to 10.5 |
| Long term stability | | | | At least |
| | | | | 12 months |

In accordance with a further aspect of the invention there are provided methods of preparing the aprepitant emulsion compositions described herein. For example, one suitable method comprises
a) providing an aqueous phase which includes water and optional ingredients such as a tonicity agent, e.g. sucrose, pH adjusting agents, e.g. oleic acid or sodium oleate, base, e.g. NaOH, buffers, other pharmaceutically acceptable excipients useful in emulsion formation;
b) providing an oil phase which includes the aprepitant, phosphatidylcholine emulsifier, e.g. soya PC, cosolvent, e.g., ethanol, and, if included, surfactant, e.g. DSPE-PEG 2000, Na, and an amount of a pH adjuster, e.g., sodium oleate or oleic acid, sufficient to provide the emulsion formed by combining the water and oil phases with an initial pH of at least 9.5;
c) combining the oil phase with the aqueous phase under conditions sufficient to form an oil in water emulsion, wherein the emulsion has an initial pH of at least 9.5; and
d) milling the emulsion optionally with a tonicity agent such as propylene glycol or sucrose, whereby a stable aprepitant emulsion composition is formed.

In some alternative aspects, the process may be modified wherein all of pH adjusting agent is preferably included only in the oil phase; and/or the surfactant is omitted from the formulation. Further alternative aspects include methods in which the surfactant is included, it is only in the aqueous phase instead of the oil phase, as well as methods in which the pH adjusting agents, e.g. sodium oleate or orleic acid, is included only in the aqueous phase of the emulsion.

A “stable” composition of the invention means a pharmaceutical composition having sufficient stability at room temperature conditions to have utility as a pharmaceutical product. Preferably, a “stable” composition of the invention has sufficient stability to allow storage at room temperature conditions, preferably between about 15° C. and about 30° C., more preferably about 20° C. to about 25° C., most preferably about 25° C., and between about 55% to about 65% RH (e.g., about 60% RH), for a reasonable period of time, e.g., the shelf-life of the product which can be as short as one month but is typically three months or longer.

For purposes of the present invention, a “stable” composition of the invention also includes specific ranges of impurities as described herein. For example, inventive formulations will preferably contain less than 0.5% of precipitate Aprepitant and in some embodiments, product will have no precipitated aprepitant after 2-12 months at room temperature and standard relative humidity ranges mentioned above as observed by sediment at the bottom of the vial. Preferably, a “stable” composition is one which has minimal degradation of the aprepitant, e.g., it retains at least about 90% of un-degraded active, preferably at least about 95%, more preferably at least about 99%, after storage at about 15-30° C. for a 1 to 3 year period of time. Stable composition also implies that composition retains physical stability with globule size, zeta potential, particulate matter within the specified limits, mean globule size of <200 nm, −10 to −90 Zeta potential and particulate matter complying with USP limits for small and large volume parenterals.

### EXAMPLES

Materials: Aprepitant (>99% purity) was procured from Glenmark Lifesciences Ltd, India Phosphatidyl Choline (Soya and egg), Soya bean oil, 1,2-Distearoyl-sn-glycero-3-phosphoethanolamine-Poly(ethylene glycol) (DSPE-PEG₂₀₀₀) were procured from Lipoid GMBH, Germany. Sodium oleate was procured from three different sources, Lobachem, India, Lipoid Germany, and Doosan, South Korea. Oleic acid was procured from Croda, USA. All other chemicals and reagents were of high purity grade and were procured from reputable local vendors.

### Methods

### HPLC Method:

The method reported in USP for determination of organic impurities of Aprepitant capsule was used for assessing potency during stability testing of samples of Aprepitant in emulsion. Method is as described below.

### Mobile Phase Preparation

Dilute phosphoric acid: Dilute 1 mL of phosphoric acid with water to 1 L.
Solution A: Acetonitrile and Dilute phosphoric acid (5:95)
Solution B: Acetonitrile and Dilute phosphoric acid (95:5)
Diluent: Isopropyl alcohol. This suits emulsions. Isopropyl alcohol dissolves oil.

### Preparation of Standard Solution:

Aprepitant 0.0012 mg/mL in diluent.

### Sample Preparation:

Appropriate volume of sample diluted with diluent to get nominal concentration of 0.6 mg/mL of Aprepitant.

### Chromatographic Conditions:

Flow rate: 1.0 mL/min, Column: 4.6-mm 15-cm; 5-μm packing L1.
Column temperature: 35° C., Injection volume: 10 μL Detection: 210 nm

TABLE 2
Gradient program for HPLC method

| Time (Minutes) | % Mobile phase A | % Mobile phase B |
|---|---|---|
| 0 | 60 | 40 |
| 20 | 58 | 42 |
| 25 | 35 | 65 |
| 33 | 35 | 65 |
| 34 | 60 | 40 |
| 45 | 60 | 40 |

### Example 1: Effect of Inclusion of Sodium Benzoate to Emulsion Formulation

TABLE 3
Formulation composition of Form-81 with sodium Benzoate

Form-81

| Ingredient | Quantity (gm) | % W/W |
|---|---|---|
| Aprepitant | 0.13 | 0.72 |
| Egg PC | 1.85 | 10.28 |
| Ethanol | 0.5 | 2.78 |
| Soya bean oil | 1.7 | 9.45 |
| DSPE-PEG-Sod | 0.1 | 0.56 |
| Sod. Oleate | 0.1 | 0.56 |
| (Loba Chemie Pvt Ltd) | | |
| Purified Water | 13.533 | 75.24 |
| Sod. Benzoate | 0.083 | 0.46 |
| Total | 17.99 | 100.0 |

### Process

Oil phase: Soya PC dissolved in ethanol by stirring, oil was added to this mixture and mixed to get clear solution. The solution was heated to about 55-68° C., while stirring aprepitant was added and continued stirring until all of the drug is dissolved. After cooling to room temperature, Sodium oleate was added to mixture and stirred to get uniform dispersion. Ethanol quantity adjusted by adding ethanol to mixture to compensate loss. Weight of container was recorded at each stage.

### Water Phase:

DSPE-PEG Sodium, Sodium Benzoate were added to water and stirred to get clear solution.

### Coarse Emulsion Preparation:

Oil phase slowly added to water phase slowly while water phase stirred at 1000 RPM using overhead stirrer.

### Microfluidization:

Coarse emulsion was milled using microfluidizer (Microfluidizer LM-25/30), 1 pass at 10 PSI, 1 pass at 20 PSI and 10-15 pass at 25 PSI.

TABLE 4
Observations of Sodium Oleate effects on emulsion formulation

| | Sodium Oleate from | Sodium Oleate from | Sodium Oleate from |
|---|---|---|---|
| Parameter | Lipoid, Germany | Loba Chemie, India | Doosan, South Korea |
| pH of 5.55 | 10.36 | 10.78 | 10.30 to 10.45 |
| mg/mL solution | | | |
| Clarity | Hazy solution | Clear solution | Clear solution |
| Emulsion | Emulsion formed after | Emulsion formed after | Emulsion formed after |
| formation | mixing oil and water | mixing oil and water | mixing oil and water |
| | phases and stirring. | phases and stirring. | phases and stirring. |
| | Emulsion is slightly | Free flowing emulsion. | Emulsion is slightly |
| | viscous and after passing | | viscous and after passing |
| | through microfluidizer, it | | through microfluidizer, |
| | is free flowing. | | it is free flowing. |

### Inferences:

Inclusion of Sodium Benzoate has profound effect on formation of emulsion. It appears that change of oleate source has moderate impact on emulsion formation. Although oleate from Loba Chemie leads to free-flowing emulsion after mixing water and oil phase, slightly viscous emulsions were formed when Oleate from Doosan and Lipoid were used. There was no cream like mass formed with any of the oleate. Addition of sodium benzoate to water phase could have led to good emulsion formation even with the oleate from Doosan and Lipoid. The oil dispersing property of benzoate would have aided emulsion formation.

### Example 2: Effect of Benzoate on Viscosity of Emulsion Formulations

TABLE 5
Formulation composition with Vitamin
E TPGS and Sodium Benzoate

| | 40009-039 | 40009-040 |
|---|---|---|
| Ingredient | mg/mL | mg/mL |
| Aprepitant | 7.22 | 7.22 |
| Egg Lecithin | — | — |
| Purified Egg | 102.7 | 102.7 |
| phosphatidylcholine | | |
| Ethanol | 27.78 | 27.78 |
| Soybean oil, USP | 88.89 | 88.89 |
| Vit-E-TPGS | 5.5 | 5.5 |
| Sodium oleate | 5.5 | 5.5 |
| Sodium Benzoate | 0.0 | 4.62 |
| Sucrose | 55.56 | 55.56 |
| 0.1M NaOH solution | 14.02 | 14.02 |
| Purified water | 693 | 688 |

### Process

Step 1: Water was taken in beaker, dilute NaOH solution (0.1 M) was added to beaker and Vitamin E TPGS was added to beaker. The mixture was stirred using magnetic stirrer and bead at about 500-800 RPM and solution was heated and maintained at 40° C. during stirring to dissolve TPGS. Sucrose was added to this mixture and mixed to dissolve.

Note: In all next batches, Sucrose was added after oil and water phases were mixed and coarse emulsion was formed.

Step-2: In another beaker, Egg Phosphatidylcholine and Ethanol were weighed into, weight of beaker with ethanol, Egg PC and Magnetic bead was noted down. Mixture was stirred at room temperature at 500 RPM using magnetic stirrer and stirring was continued until clear solution was formed. Ethanol loss due to evaporation was monitored by weighing at each stage and loss was compensated by addition of ethanol.

Step-3: Add Soya bean oil to step-2 mixture in beaker and mixed at 500 RPM using magnetic stirrer for 3 minutes.

Step-4: Solution of step-3 was heated to 65-70° C. and Aprepitant was added. Mixture was stirred using magnetic stirrer and bead till clear solution was formed (1000 RPM, 68.5° C.). The weight of beaker before addition of Aprepitant was noted and exact weight of Aprepitant was noted. At end of the process, weight loss due to evaporation of ethanol was compensated by adding ethanol.

Step-5: Sodium Oleate was added to mixture of step-5 and mixture was stirred for 20 minutes at 1500 RPM using overhead stirrer.

Step-6: Aqueous phase (Step-1) was added slowly to oil phase (Step-6) under continuous stirring at 1500 RPM using overhead stirrer (About 5 minutes taken to add all of aqueous phase) and mixture stirred at same speed for 20 minutes to get coarse emulsion which was free flowing.

Step-7: Coarse emulsion of step-7 was milled using Microfluidizer (LM-20) following cycles: 1 cycle at 10000 PSI, 1 cycle at 20000 PSI and 5 cycles at 25000 PSI.

### Inference:

The formulation without Sodium Benzoate showed higher viscosity, 40009-039 has 8 times more viscosity than 40009-040. Higher viscosity bound to cause problems for syringeability and injectability.

In addition, inclusion of sodium benzoate has profound effect on ease of the process. The number of cycles required to attain globule size of around 100 nm.

This positive effect of Sodium Benzoate is quite surprising and unknown.

Results shown are in

TABLE 6
Results of 40009-039 and 040

| Parameter | 40009-039 | 40009-040 |
|---|---|---|
| Viscosity, cps | 38.15 | 4.99 |
| Number of cycles at | 5 cycles | 3 cycles |
| 25000 PSI needed to bring | | |
| mean globule size to | | |
| around 100 nm | | |

### Example-3: Effect of Surfactant Concentration on the Physical Stability of Emulsion

TABLE 7
Composition for B. No: 40009-009, 011 and 40009-015

| | 40009-011 | 40009-009 | 40009-015 |
|---|---|---|---|
| Ingredient | Qty/mL | Qty/mL | Qty/mL |
| Aprepitant | 7.22 | 7.22 | 7.22 |
| Purified Egg phosphatidylcholine | 102.87 | 107.22 | 108.3 |
| (Lipoid E PC S) | | | |
| Ethanol | 27.78 | 27.78 | 27.78 |
| Soya bean oil, USP | 88.89 | 88.89 | 88.80 |
| Vit-E-TPGS | 5.56 | 1.11 | 0.0 |
| Sodium Oleate | 5.56 | 6.67 | 6.66 |
| (Lipoid) | | | |
| Sodium Benzoate | 0.0 | 4.63 | 4.63 |
| Sucrose | 55.56 | 55.56 | 55.50 |
| 0.1M NaOH solution | 14 | 14 | 14 |
| Purified Water | 665.00 | 686.91 | 687.0 |
| 0.1M NaOH solution | 14 | 14 | 14 |

Procedure: Same as 40003-039 and 40.

### Results:

TABLE 8
Results of formulations 40009-009-015

| | TPGS | |
|---|---|---|
| Formulation | (Mg/mL) | Results |
| 40009-015 | 0 | No Precipitation after |
| | | 12 months at 15° C., |
| | | no particle >0.3 μm |
| | | found in size histogram |
| 40009-009 | 1.1 | No Precipitation after |
| | | 9 months at 15° C., |
| | | no particle >0.3 μm |
| | | found in size histogram |
| 40009-011 | 5.5 | Precipitation observed |
| | | after 8-9 months at 15° C., - with |
| | | particles >18-20 μm |
| | | found in size histogram |

### Inference:

The presence of higher amount of surfactant, Vitamin TPGS in the formulation leads to precipitation of Aprepitant in the formulation. This is quite in contrast with established trend where higher amount of surfactant stabilizes emulsion. However, removal of surfactant leads to requirement of higher phospholipids.

### Example 4: Formulations with 10% W/W of Phosphatidylcholine, Sodium Oleate and No Surfactants

TABLE 9
Compositions for formulations with 10% W/W egg phosphatidylcholine and sodium oleate

| | 40009- | | 40003- | | 40009- | | 40009- | | 40009- | | 40009- | | 40009- | |
|---|---|---|---|---|---|---|---|---|---|---|---|---|---|---|
| Ingredients | 062 | | 063 | | 099 | | 103 | | 104 | | 106 | | 108 | |
| Aprepitant | 7.22 | mg | 7.22 | mg | 7.22 | mg | 7.22 | mg | 7.22 | mg | 7.22 | mg | 7.22 | mg |
| Egg | 102.7 | mg | 100 | mg | 100 | mg | 100 | mg | 100 | mg | 100 | mg | 101 | mg |
| Phosphatidyl- | | | | | | | | | | | | | | |
| choline | | | | | | | | | | | | | | |
| Ethanol | 27.7 | mg | 27.7 | mg | 27.7 | mg | 27.7 | mg | 27.7 | mg | 27.7 | mg | 27.7 | mg |
| Soyabean oil | 88.8 | mg | 88.8 | mg | 88.8 | mg | 88.8 | mg | 88.8 | mg | 88.8 | mg | 88.8 | mg |
| Sodium Oleate | 6.66 | mg | 6.66 | mg | 8 | mg | 8 | mg | 8 | mg | 8 | mg | 8 | mg |
| 0.1M NaOH | 14 | mg | 14 | mg | 14 | mg | 18 | mg | 18 | mg | 25 | mg | 25 | mg |
| solution | | | | | | | | | | | | | | |
| Sodium | 4.62 | mg | 4.62 | mg | 4.62 | mg | 4.62 | mg | 5 | mg | 5 | mg | 5 | mg |
| Benzoate | | | | | | | | | | | | | | |
| Sucrose | 55.5 | mg | 55.5 | mg | 55.5 | mg | 55.5 | mg | 55.5 | mg | 55.5 | mg | 55.5 | mg |
| Milli-Q-water | 692.5 | mg | 695.3 | mg | 693.88 | mg | 689.95 | mg | 689.5 | mg | 682.5 | mg | 681.5 | mg |

### Process:

Step 1: Water was taken in beaker, added Sodium benzoate, 0.1 M sodium hydroxide and sucrose to beaker and stirred using magnetic stirrer and bead at 500 RPM for 05 min to get clear colorless solution.

Step-2: In another beaker, Egg Phosphatidylcholine and Ethanol were weighed and stirred using magnetic stirrer and bead at 500 RPM for 21 min to get clear amber color solution. At the end of mixing, beaker with bead was weighed and loss of weight was compensated with addition of ethanol.

Step-3: Added Soya bean oil to step-2 mixture in beaker and mixed at 500 RPM using magnetic stirrer for 3 minutes.

Step-4: Solution of step-3 was heated to 65-70° C. and Aprepitant was added. Mixture was stirred using magnetic stirrer and bead till clear solution was formed (500 RPM, 69.5° C.). Time taken 13 minutes. The weight of beaker before addition of Aprepitant was noted and exact weight of Aprepitant was noted. At end of the process, weight loss due to evaporation of ethanol was compensated by adding ethanol.

Step-5: Sodium Oleate was added to mixture of step-5 and mixture was stirred for 14 minutes at 1500 RPM using overhead stirrer.

Step-6: Aqueous phase (Step-1) was added slowly to oil phase (Step-5) under continuous stirring at 1500 RPM using overhead stirrer (About 3 minutes taken to add all of aqueous phase) and mixture stirred at same speed for 10 minutes to get coarse emulsion.

Step-7: Coarse emulsion of step-6 was milled using Microfluidizer (LM-20) following cycles: 1 cycle at 10K PSI, 1 cycle at 20K PSI, 5 cycles at 25K PSI

TABLE 10
Physical parameters of formulations 40009-062 to 40009-108

| | 40009- | 40003- | 40009- | 40009- | 40009- | 40009- | 40009- |
|---|---|---|---|---|---|---|---|
| Parameters | 062 | 063 | 099 | 103 | 104 | 106 | 108 |
| pH | Not done | Not done | 9.18 | 9.07 | 9.23 | 9.5 | 9.45 |
| Time taken for | 12 days | 12 days | 12 days | 15 days | 12 days | 22 days | 1 Month |
| appearance of | | | | | | | |
| precipitate when | | | | | | | |
| stored at 40° C./75% | | | | | | | |
| RH | | | | | | | |

### Results

Inference: The formulations with about 10% W/W phosphatidylcholine and sodium oleate did not show adequate physical stability. All the formulations tested showed precipitation within 12-30 days and precipitate settles at the bottom of the vials. It was also noted that, changing oleate source from Lipoid, Germany to Doosan, Korea did not improve the stability, precipitation was observed.

### Example 5: Formulations with 10% W/W of Phosphatidylcholine, Oleic Acid and No Surfactants

TABLE 11
Compositions of the formulations 40009-100 to 40009-105

| | 40009- | | 40009- | | 40009- | | 40009- | |
|---|---|---|---|---|---|---|---|---|
| Ingredients | 100 | | 101 | | 102 | | 105 | |
| Aprepitant | 7.22 | mg | 7.22 | mg | 7.22 | mg | 7.22 | mg |
| Egg | 100 | mg | 100 | mg | 100 | mg | 100 | mg |
| Phosphatidylcholine | | | | | | | | |
| Ethanol | 27.7 | mg | 27.7 | mg | 27.7 | mg | 27.7 | mg |
| Soyabean oil | 88.8 | mg | 88.8 | mg | 88.8 | mg | 88.8 | mg |
| Oleic acid | 6.66 | mg | 6.66 | mg | 5.32 | mg | 5.32 | mg |
| NaOH | 0.94 | mg | 1 | mg | 1 | mg | 1.2 | mg |
| Sodium Benzoate | 4.62 | mg | 4.62 | mg | 4.62 | mg | 5 | mg |
| Sucrose | 55.5 | mg | 55.5 | mg | 55.5 | mg | 55.5 | mg |
| Milli-Q-water | 708.3 | mg | 708.3 | mg | 709.6 | mg | 709 | mg |

### Process

Step 1: Water was taken in beaker, added NaOH, Sodium benzoate, sucrose were added to beaker and stirred using magnetic stirrer and bead at 500 RPM for 06 min to get clear colorless solution.

Step-2: Take another beaker, weigh Oleic acid into this beaker. To this transferred step 1 solution with continuous stirring using magnetic stirrer using bead at 1000 RPM for 07 min to get clear colorless solution.

Step-3: In another beaker, Egg Phosphatidylcholine and Ethanol were weighed and stirred using magnetic stirrer and bead at 500 RPM for 21 min to get clear amber color solution. At the end of mixing, beaker with bead was weighed and loss of weight was compensated with addition of ethanol.

Step-4: Add Soya bean oil to step-3 mixture in beaker and mixed at 500 RPM using magnetic stirrer for 3 minutes.

Step-5: Solution of step-4 was heated to 65-70° C. and Aprepitant was added.

Mixture was stirred using magnetic stirrer and bead till clear solution was formed (500 RPM, 72.6° C.). Time taken 12 minutes. The weight of beaker before addition of Aprepitant was noted and exact weight of Aprepitant was noted. At end of the process, weight loss due to evaporation of ethanol was compensated by adding ethanol.

Step-6: Aqueous phase (Step-2) was added slowly to oil phase (Step-5) under continuous stirring at 1500 RPM using overhead stirrer (About 2 minutes taken to add all of aqueous phase) and mixture stirred at same speed for 10 minutes to get coarse emulsion. which was free flowing.

Step-7: Coarse emulsion of step-6 was milled using Microfluidizer (LM-20) following cycles: 1 cycle at 10K PSI, 1 cycle at 20K PSI, 4 cycles at 25K PSI

### Results

TABLE 12

| Parameters | 40009-100 | 40003-101 | 40009-102 | 40009-105 |
|---|---|---|---|---|
| pH** | 8.57 | 9.05 | 9.77 | 10.28 |
| Time taken for appearance | Precipitate | Precipitate | Precipitate | No precipitation |
| of precipitate when stored | after 7 days | after 7 days | after 15 days | observed till 3 |
| at 40° C./75% RH | | | | months |

**pH measured immediately after preparation, within 2-3 hours of final emulsion was made.

### Inference:

Physically stable formulation is achieved with oleic acid in the formulation (40009-105). The oleic acid is of high purity grade, it has >85% of oleic acid and >96% of total fatty acids. High fatty acid content along with higher pH leads to greater physical stability with the formulation stable up to three months at 40° C./75% RH.

### Example 5: Formulations with 10% W/W of Phosphatidylcholine and Varying Concentration of Oleic Acid

TABLE 13
Compositions of formulations, varying concentration of Oleic acid

| Ingredients | 40009-105 | | 40009-107 | | 40009-122 | | 40009-123 | | 40009-124 | |
|---|---|---|---|---|---|---|---|---|---|---|
| Aprepitant | 7.22 | mg | 7.22 | mg | 7.22 | mg | 7.22 | mg | 7.22 | mg |
| Egg | 100 | mg | 100 | mg | 100 | mg | 100 | mg | 100 | mg |
| Phosphatidylcholine | | | | | | | | | | |
| Ethanol | 27.7 | mg | 27.7 | mg | 27.7 | mg | 27.7 | mg | 27.7 | mg |
| Soyabean oil | 88.8 | mg | 88.8 | mg | 88.8 | mg | 88.8 | mg | 88.8 | mg |
| Oleic acid | 5.32 | mg | 6.66 | mg | 7 | mg | 8 | mg | 4.73 | mg |
| NaOH | 1.20 | mg | 1.5 | mg | 1.5 | mg | 1.5 | mg | 1.2 | mg |
| Sodium Benzoate | 4.62 | mg | 4.62 | mg | 4.62 | mg | 4.62 | mg | 5 | mg |
| Sucrose | 55.5 | mg | 55.5 | mg | 55.5 | mg | 55.5 | mg | 55.5 | mg |
| Milli-Q-Water | 709.35 | mg | 707.75 | mg | 707.75 | mg | 707.75 | mg | 709 | mg |

Process: Same as 40009-105 mentioned above. For 40009-107, Oleic acid was added to oil phase after step-5 (instead of to the aqueous phase), just before the mixing of oil and water phase.

### Results

TABLE 14

| Parameters | 40009-105 | 40009-107 | 40009-122 | 40009-123 | 40009-124 |
|---|---|---|---|---|---|
| pH | 10.55 | 10.31 | 10.18 | 9.866 | 9.006 |
| Time taken for | Clear till | Clear till | Precipitate | Precipitate | Precipitate |
| precipitation at | three months | three months | appeared | appeared | appeared |
| 40° C./75% RH | | | within one | within one | after 2.5 |
| | | | month | month | months |

### Inference:

The quantity of Oleic acid in the formulation is very critical. Both higher and lower amounts of oleic acid leads to precipitation. Oleic acid between 5.3 to 6.66 provided the best results.

TABLE 15
Stability of selected formulation:

| | 40009-105 | | |
|---|---|---|---|
| Test Parameter | Initial | 3M, 25° C./60% RH | 6M, 25° C./60% RH |
| Description | Opaque, off-white to | Opaque, off-white to | Opaque, off-white to |
| | pale yellowish | pale yellowish | pale yellowish |
| | emulsion. | emulsion. | emulsion. |
| pH (0.9% NaCl) | 9.439 | 8.258 | 8.043 |
| pH (5% Dextrose) | 9.378 | 9.055 | 8.952 |
| pH (Water) | 10.21 | 9.381 | 9.187 |
| pH (Direct) | 10.141 | 8.613 | 8.355 |
| Osmolality (mOsm/KG) with | 359 | 365 | 369 |
| 0.9% NaCl | | | |
| Osmolality (mOsm/KG) with | 375 | 374 | 379 |
| 5% Dextrose | | | |
| Particle size (Z-Avg) in nm | 104.3 | 103.5 | 108.5 |
| Zeta Potential (mv) | −56.87 | −59.10 | −56.90 |
| Assay (%) | 98.5 | 99.2 | 97.4 |
| Ethanol content (%) | 87.3 | Not done | 77.9 |
| Desfluoro Aprepitant (%) | ND | ND | ND |
| Aprepitant diastereomers | ND | ND | ND |
| (RRR & RSS) (%) | | | |
| Any unspecified impurity (%) | ND | ND | ND |
| Total impurities (%) >0.1% | ND | ND | ND |
| Total impurities (%) All | ND | ND | ND |

### Inference:

Formulation 40009-105 is stable for at least for 6 months at 25° C./60% RH

### Example 6: Effect of Final Ethanol Concentration in the Formulation on Stability of the Formulations

TABLE 16
Composition of formulations with varying amount of ethanol

| Ingredients | 40009-105 | | 40009-107 | | 40009-111 | | 40009-110 | |
|---|---|---|---|---|---|---|---|---|
| Aprepitant | 7.22 | mg | 7.22 | mg | 7.22 | mg | 7.22 | mg |
| Egg | 100 | mg | 100 | mg | 100 | mg | 100 | mg |
| Phosphatidylcholine | | | | | | | | |
| Ethanol | 24.18 | mg | 22.4 | mg | 16.67 | mg | 18.25 | mg |
| Soyabean oil | 88.8 | mg | 88.8 | mg | 88.8 | mg | 88.8 | mg |
| Oleic acid | 5.32 | mg | 6.66 | mg | 5.32 | mg | 6.66 | mg |
| NaOH | 1.20 | mg | 1.5 | mg | 1.25 | mg | 1.5 | mg |
| Sodium Benzoate | 4.62 | mg | 4.62 | mg | 4.62 | mg | 4.62 | mg |
| Sucrose | 55.5 | mg | 55.5 | mg | 55.5 | mg | 55.5 | mg |
| Milli-Q-Water | 709.35 | mg | 707.75 | mg | 709.35 | mg | 707.75 | mg |

Process: Same as 40009-105 mentioned above. For 40009-107, Oleic acid was added to oil phase after step-5, just before the mixing of oil and water phase. The formulations, 40003-110 and 4003-111, after step-5 oil was mixed 1500 RPM for 20 minutes.
Results: Given in Tables 17-20

TABLE 17
Physicochemical observations

| Parameter | 40009-105 | 40009-107 | 40009-111 | 40009-110 |
|---|---|---|---|---|
| Ethanol content initial | 24.18 | 22.4 | 16.67 | 18.25 |
| (Mg/mL) | | | | |
| Physical observation | No | No | No | No |
| after 6 months at | precipitation | precipitation | precipitation | precipitation |
| 25° C./60% RH | | | | |
| Physical observation | Gel-like | Gel-like | No | No |
| after 12 months at | precipitation at | precipitation | precipitation | precipitation |
| 25° C./60% RH | the bottom | at the bottom | | |

TABLE 18
Stability data for 40009-105 and 40009-111

| | 40009-105 | | | 40009-111 | |
|---|---|---|---|---|---|
| | | 6 M, | 12 M, | 6 M, | 12 M, |
| | | 25° C./ | 25° C./ | 25° C./ | 25° C./ |
| | Initial | 60% RH | 60% RH | 60% RH | 60% RH |
| Description | Opaque, off- | Opaque, off- | Formulation | Opaque, off- | |
| | white to pale | white to pale | showed gel like | white to pale | |
| | yellowish | yellowish | precipitate at | yellowish | |
| | emulsion | emulsion | the bottom, | emulsion | |
| pH (0.9% NaCl) | 9.439 | 8.043 | except assay | 8.054 | 7.556 |
| pH (5% Dextrose) | 9.378 | 8.952 | no other | 8.969 | 8.672 |
| pH (Water) | 10.21 | 9.187 | parameters | 9.352 | 9.015 |
| pH (Direct) | 10.141 | 8.355 | tested | 8.425 | 8.14 |
| Osmolality | 359 | 369 | | 349 | 331 |
| (mOsm/KG) with | | | | | |
| 0.9% NaCl | | | | | |
| Osmolality | 375 | 379 | | 360 | 338 |
| (mOsm/KG) with | | | | | |
| 5% Dextrose | | | | | |
| Particle size (Z-Avg) | 104.3 | 108.5 | | 108.2 | |
| in nm | | | | | |
| Zeta Potential (mv) | −56.87 | −56.90 | | −58.91 | |
| Assay (%) | 98.5 | 97.4 | Formulation | 102.3 | 95.9 |
| Ethanol content (%) | 87.3 | 77.9 | showed gel like | 59.9 | Not done |
| Desfluoro | ND | ND | precipitate at | ND | ND |
| Aprepitant (%) | | | the bottom, | | |
| Aprepitant | ND | ND | except assay | ND | ND |
| diastereomers (RRR | | | no other | | |
| & RSS) (%) | | | parameters | | |
| | | | tested | | |
| Any unspecified | ND | ND | ND | ND | ND |
| impurity (%) | | | | | |
| Total impurities (%) >0.1% | ND | ND | ND | ND | ND |
| Total impurities (%) | ND | ND | ND | ND | ND |
| All | | | | | |

TABLE 19
Stability data for 40009-107 and 40009-110

| | 40009-107 | | 40009-110 | |
|---|---|---|---|---|
| | 6 M, | 12 M, | 6 M, | 6 M, |
| Test Parameter | 25° C./60% RH | 25° C./60% RH | 25° C./60% RH | 25° C./60% RH |
| Description | Opaque, off-white | Formulation | Opaque, off-white to | Opaque, off- |
| | to Amber color | showed gel like | Amber color | white to Amber |
| | emulsion in a clear | precipitate at the | emulsion in a clear | color emulsion |
| | glass vial closed | bottom, except | glass vial closed with | in a clear glass |
| | with a rubber | assay no other | a rubber stopper and | vial closed with |
| | stopper and | parameters tested | crimped with an | a rubber |
| | crimped with an | | aluminum seal | stopper and |
| | aluminum seal | | | crimped with |
| | | | | an aluminum |
| | | | | seal |
| pH (0.9% NaCl) | 8.159 | | 8.169 | 7.794 |
| pH (5% Dextrose) | 9.079 | | 9.106 | 8.753 |
| pH (5% water) | 9.426 | | 9.407 | 9.103 |
| pH (Direct) | 8.484 | | 8.483 | 8.053 |
| Osmolality | 366 | | 355 | 337 |
| (mOsm/KG) with | | | | |
| 0.9% NaCl | | | | |
| Osmolality | 376 | | 367 | 357 |
| (mOsm/KG) with | | | | |
| 5% Dextrose | | | | |
| Particle size (Z- | 105.7 | | 109.9 | Not done |
| Avg) in nm | | | | |
| Zeta Potential | −57.93 | | −60.66 | Not done |
| (mv) | | | | |
| Assay (%) | 98.8 | 69.7 | 101.2 | 95.7 |
| Ethanol content | 73.7 | Not Done | 65.3 | Not done |
| (%) | | | | |

Organic Impurities (%)

| | | | | |
|---|---|---|---|---|
| Desfluoro | ND | ND | ND | ND |
| Aprepitant (%) | | | | |
| Aprepitant | ND | ND | ND | ND |
| diastereomers | | | | |
| (RRR & RSS) (%) | | | | |
| Any unspecified | ND | ND | ND | ND |
| impurity RRT- | | | | |
| 0.44 (%) | | | | |
| Total impurities | ND | ND | ND | ND |
| (%)[Reporting | | | | |
| threshold 0.1% | | | | |
| applied in the | | | | |
| summation] | | | | |
| Total impurities | ND | ND | ND | ND |
| (%)[Reporting | | | | |
| threshold 0.1% | | | | |
| not applied in the | | | | |
| summation] | | | | |

TABLE 20
Comparative stability of formulations at 25° C./60% RH

| Parameter | 40009-105 | 40009-105 | 40009-110 | 40009-111 | Cinnvanti ® |
|---|---|---|---|---|---|
| Stability at | Stable for | Stable for | Stable for | Stable for | Stable for 2 months |
| 250° C./60% RH | 6 months | 6 months | 12 months | 12 months | As per product information |
| | | | | | leaflet, product should not |
| | | | | | be stored at room temperate |
| | | | | | more than 60 days |

### Inference:

The amount of ethanol in the formulation has a significant effect on the stability of the formulation.

Formulation with <18 mg/ml of ethanol provides stability of at least 12 months at 25° C./60% RH. RLD, the Cinnvanti® is only stable for about 2 months at 25° C./60% RH. The formulation 40009-110 and 40009-111 provide distinct advantage over Cinnvanti® in terms of stability at 25° C./60% RH.

Another interesting observation is that formulation when 10% or less of phosphatidylcholine is used, physically stable formulations can only be formed with oleic acid or sodium oleate with higher amount of Oleic acid is used as stabilizer coupled with initial pH>10. Oleic acid from Croda and Sodium Oleate from Loba chemie produced stable emulsion with phosphatidylcholine at 10% W/W. Interestingly, both of these materials had >80% of oleic acid content. In addition presence of other fatty acids play role in stability and higher amount of linoleic acid might not be good for stability as in case of Doosan Sodium Oleate.

TABLE 21
Comparison of Oleic and Oleates from different manufacturer.

| | Sodium Oleate | Sodium Oleate | Sodium Oleate B | |
|---|---|---|---|---|
| | Extra pure | Lot: SDIC26 | Lot: 520400- | Super Refined |
| | Lot: 314791910, | Doosan Solus, | 2210895-01 | Oleic Acid |
| | Loba Chemie, | Republic of | Lipoid, | Lot: 090000399848 |
| Parameter | India | Korea, | Germany | Croda, USA |
| Assay of Sodium | 99 | 79 | Not mentioned in | Not applicable |
| Oleate | | | COA | |
| Oleic acid content | 82% | 82% | 57.6 | 86.4 |
| (% of Sodium | | | | |
| Oleate) | | | | |
| Linoleic acid | — | 11.8% | Not mentioned in | 6.4 |
| (% of Sodium | | | COA | |
| Oleate) | | | | |
| Lauric acid | — | 1.8% | Not mentioned in | Not present |
| (% of Sodium | | | COA | |
| Oleate) | | | | |
| Palmitic acid | — | 4.9% | Not mentioned in | 2.9 |
| (% of Sodium | | | COA | |
| Oleate) | | | | |
| Free alkali (As | 0.5% | Not present | Not mentioned in | Nil |
| NaOH) | | | COA | |

### Example 6: Effect of Addition of Oleic Acid Formulation on Dissolution Aprepitant from the Emulsion and Pharmacokinetic Profile of the Aprepitant in Wistar Rats

While screening formulations for pharmacokinetic profile in Wistar Rats, the formulations with different globule size tested. The two sets of formulations, one where the oleic acid was added to oil phase and another with oleic acid added to water were screened and provided two different trends which were contrary to expectations. Results summarized here.

TABLE 22
Formulation compositions tested for Rat pharmacokinetic profile.

| | 40009-131 | 40009-132 | 40009-139 | 40009-142 | 40009-143 |
|---|---|---|---|---|---|
| Ingredients | Oleic acid added to water phase | | | Oleic acid added to oil phase | |
| Aprepitant | 7.22 | 7.22 | 7.22 | 7.22 | 7.22 |
| Egg Phosphatidylcholine | 100 | 100 | 100 | 100 | 100 |
| Ethanol | 27.7 | 27.7 | 27.7 | 27.7 | 27.7 |
| Soyabean oil | 88.8 | 88.8 | 88.8 | 88.8 | 88.8 |
| Oleic acid | 5.32 | 5.32 | 5.32 | 5.32 | 5.32 |
| NaOH | 1.2 | 1.2 | 1.2 | 1.2 | 1.2 |
| Sodium Benzoate | 5 | 5 | 5 | 5 | 4.62 |
| Sucrose | 55.5 | 55.5 | 55.5 | 55.5 | 55.5 |
| Milli-Q-water | 709 | 709 | 709 | 709 | 709 |

Process: Same as 40009-105 with following changes.
a) Oleic acid added to water phase for the formulation 40009-131-139.
b) Oleic acid added to oil phase for the formulations 40009-142 and 40009-143.
c) After adding Oleic acid to oil phase, oil phase was mixed for 20 minutes using overhead stirrer (For 40009-142 and 40009-143).

### Procedure for Dissolution Testing:

TABLE 23
Dissolution test parameters

| Parameters | Details |
|---|---|
| Apparatus | USP Type-II, Apparatus with Paddle, RPM 100 |
| Sinker | Include part number, manufacturer, and dimensions |
| Medium | 40% V/V Acetonitrile in Phosphate Buffer pH 7.4 |
| Volume | 500 mL |
| Temperature | 37° C. ± 0.5° C. |
| Sample | Sample Volume: 1 mL formulation and 5 mL Phosphate Buffer |
| Preparation | pH 7.4 filled in dialysis tube with both ends tied tightly. |
| and Tubing | Dialysis Tubing: |
| | Dialysis tubing cellulose membrane. |
| | Manufacturer: Sigma-Aldrich (Merck) |
| | Catalogue/Part No: D9777 |
| | Flat width of tube: 25 mm |
| | Cut off diameter: 14 KDa |
| | Pre-treatment of Dialysis Tube: |
| | Required length of tube (4 inches) was dipped in water |
| | maintained at 90° C. for 10 minutes and followed by |
| | immersion in water at ambient conditions until the time |
| | of use. |
| Dissolution | 500 mL of dissolution medium was placed into each of |
| Set up | 1000 mL dissolution vessels and after the set temperature |
| | (37 ± 0.5° C.) is attained, dialysis tube with sample |
| | was placed into stainless steel coated sinker and |
| | dropped at the bottom of flask. |
| Sampling | 10 mL of sample is collected at time points: |
| Time Points | 0.5, 1, 2, 3, 4, 5, 6 and 8 hours. |

### Procedure for Rat Pharmacokinetic Studies

TABLE 24
Details of Rat Pharmacokinetic studies.

| | |
|---|---|
| Title | A Single Dose Intravenous Pharmacokinetic (PK) Bridging Study in the Rat |
| | Comparing Inventive Aprepitant Injectable emulsion formulations with the |
| | Reference Listed Drug (RLD), Cinvanti ®, 130 mg/18 mL. |
| Species | Wistar Rats, 180 to 220 g |
| Species Control | Maintain rats under controlled environmental conditions of temperature, |
| | relative humidity, and a 12-hour light/dark cycle. |
| | Allow free access to a standard rodent diet. |
| | Tap water to available ad libitum. |
| Dose | 10 mg/kg |
| Route of Administration | IV Bolus after dilution with saline (3 mL formulation +7 mL saline) |
| Site of Injection | Tail vein injection |
| Blood sampling | Withdraw about 0.35 mL of blood from retro-orbital plexus at 0, 0.083, 0.25, |
| | 0.5, 1, 2, 4 and 6 hours. Sacrifice the animals immediately after 6-hour sample |
| | is collected. |
| Plasma processing and | Plasma separated from blood collected at each time point. The Plasma was then |
| analysis | mixed with methanol to precipitate proteins and centrifuged. Supernatant was |
| | collected and analyzed by HPLC. |

### Results: Below

TABLE 25
Pharmacokinetic data for the formulations
made by adding oleic acid to water phase

| | 40009-131C | 40009-132, | 40009-131B, | 40009-139, |
|---|---|---|---|---|
| Parameters | 82.2 nm | 89.05 nm | 102.9 nm | 111.7 nm |
| % Ethanol content (% W/W) | 88.6 | 59.1 | 88.6 | 70.7 |
| % Dissolved at 8 hours | 88.84 | 91.01 | 99.24 | 83.03 |
| Cmax Ratio, T/R*100 | 82.42 | 74.78 | 87.25 | 73.72 |
| (Against Cinvanti ®, T-Test, | | | | |
| R-Reference, Cinvanti ®) | | | | |
| AUC Ratio, T/R*100 | 73.03 | 67.26 | 67.63 | 63.94 |
| (Against Cinvanti ®, T-Test, | | | | |
| R-Reference, Cinvanti ®) | | | | |

TABLE 25
Pharmacokinetic data for the formulations made by adding oleic acid to oil phase

| | 40009-142D | 40009-143C | 40009-143B | 40009-143A |
|---|---|---|---|---|
| Parameters | 80 nm | 87.48 nm | 99.99 nm | 113 nm |
| | 48.6 | 71.4 | | |
| % Dissolved at 8 hours | 79.11 | 84.53 | 85.18 | 86.26 |
| Cmax Ratio, T/R*100 | 75.58 | 111.26 | 112.2 | 87.69 |
| (Against Cinvanti ®, T- | | | | |
| Test, R-Reference, | | | | |
| Cinvanti ®) | | | | |
| AUC Ratio, T/R*100 | 104.58 | 96.49 | 96.44 | 99.54 |
| (Against Cinvanti ®, T- | | | | |
| Test, R-Reference, | | | | |
| Cinvanti ®) | | | | |

### Observations:

a) When oleic acid was added to water phase with sodium hydroxide in it, the sodium oleate formed in-situ provides solubilizing power, acting as surfactant and causing higher dissolution at the end of 8 hours, close to 90% (40009-131-40009-139). However, AUC and Cmax ratio were low, as compared to Cinvanti®. Especially, AUC less than 75% of Cinvanti®. This was an unexpected finding.
b) On other hand, when oleic acid was added to the oil phase, dissolution was on the lower side, 79-86%. However, the AUC and Cmax ratios are higher and between 96-112% in some formulations, indicating AUC and Cmax are same as RLD, Cinvanti®. Interestingly, the AUC and Cmax increased as the mean globule size of emulsion increased. Usually, emulsions with smaller globule size release the drug faster.
c) Just way the oleic acid added to emulsion, significant effect on the performance of the product in the body. It was a surprising finding.

### Overall Observations

1. The amount of surfactant has critical role in stability emulsion, higher amount of surfactant, Vitamin E TPGS leads to precipitation after about 8-9 months when stored at 15° C. Formulations demonstrate enhanced stability when no surfactant is used. When included, however, amounts of about 0.1 to 0.15% W/W of emulsion is beneficial.
2. Oleic acid content in the Sodium Oleate or Oleic acid is very critical.
3. Finally, alkalinity of oleate is critical, to stabilize emulsion, additional alkali, sodium hydroxide is essential.
4. Inclusion of very minor amount of dilute sodium hydroxide solution in the composition results in formation of less viscous, free flowing coarse emulsion which was amenable for milling via microfluidizer.
5. Sodium Benzoate plays critical role in the formation of emulsion, it avoids formation of cream like consistency of coarse emulsion, reduces number of milling cycles. Most importantly, viscosity was reduced by 8 times compared to similar formulation without benzoate. Low viscosity is essential for syringeability and injectability of the product.
6. Surprisingly, higher alcohol destabilizes the emulsion, leading to precipitation. The ethanol content of <20 mg/mL is optimal.
7. Formulation with about 10% W/W phosphatidylcholine, needs high purity oleic acid with oleic acid content of >85% and total fatty acid content of >96%. Higher amounts sodium hydroxide is needed to form sodium oleate in-situ and maintain pH at higher side, which is essential to maintain physical stability of emulsion without precipitation.
8. Concentration of Oleic acid is very critical, about 5-6.7 mg/mL seems to be optimal, where higher and lower amount of oleic acid than this range leads to precipitation.

What is claimed:

1. An injectable aprepitant emulsion, comprising:
a) from about 0.25 to about 1.5% W/W aprepitant;
b) from about 8 to about 10.8% W/W of a phosphatidylcholine emulsifier;
c) from about 1.25 to about 2.8% W/W of a cosolvent comprising ethanol;
d) from about 7 to about 10.5% W/W of an oil;
e) from about 0.2 to about 1.0% W/W oleic acid or an oleic acid salt;
f) from about 0.25 to about 0.5% W/W sodium benzoate;
g) from about 4 to about 6% W/W of a tonicity agent selected from the group consisting of propylene glycol, sucrose and mixtures thereof;
h) from about 65 to about 80% W/W of water; and
i) from about 0.05 to about 1.5% W/W of a base;
wherein the emulsion has (i) an initial pH of greater than 9.5, (ii) a mean globule size (Z-average) of between about 60 and about 160 nm, and optionally wherein the emulsion has total impurities of less than 0.5% after 6 months at 25° at 60% relative humidity.

2. The injectable aprepitant emulsion of claim 1, wherein said formulation is free of surfactants.

3. The injectable aprepitant emulsion of claim 1, wherein the amount of aprepitant is between about 0.65 and about 0.75% W/W of the emulsion.

4. The injectable aprepitant emulsion of claim 1, wherein the phosphatidylcholine emulsifier is egg phosphatidylcholine.

5. The injectable aprepitant emulsion of claim 1, wherein the amount of phosphatidylcholine emulsifier is about 10% W/W of the emulsion.

6. The injectable aprepitant emulsion of claim 1, wherein the cosolvent is ethanol.

7. The injectable aprepitant emulsion of claim 6, wherein the amount of ethanol in the emulsion is from about 1.5 to about 1.9% W/W of the emulsion.

8. The injectable aprepitant emulsion of claim 1, wherein the oil is soya bean oil.

9. The injectable aprepitant emulsion of claim 8, wherein the amount of soya bean oil is from about 8 to about 9% W/W of the emulsion.

10. The injectable aprepitant emulsion of claim 1, wherein the amount of oleic acid is from about 0.45 to about 0.7% W/W of the emulsion.

11. The injectable aprepitant emulsion of claim 1, wherein the base is NaOH.

12. The injectable aprepitant emulsion of claim 11, wherein the amount of NaOH is from about 0.1 to about 0.5% W/W of the emulsion.

13. The injectable aprepitant emulsion of claim 1, wherein the amount of sodium benzoate is from about 0.4 to about 0.5% W/W of the emulsion.

14. The injectable aprepitant emulsion of claim 1, wherein the tonicity agent is sucrose.

15. The injectable aprepitant emulsion of claim 1, wherein the amount of tonicity agent is from about 0.5 to about 0.6% W/W of the emulsion.

16. The injectable aprepitant emulsion of claim 1, wherein the amount of water is from about 68 to about 72% W/W of the emulsion.

17. The injectable aprepitant emulsion of claim 1, wherein the emulsion is substantially free of precipitated Aprepitant.

18. The injectable aprepitant emulsion formulation of claim 1, further comprising a member of the group consisting of pH modifiers, buffering agents, antioxidants, and preservatives.

19. The injectable aprepitant emulsion formulation of claim 1, wherein the tonicity agent is sucrose.

20. The injectable aprepitant emulsion formulation of claim 1, wherein the initial pH of the composition is greater than or equal to at least 9.55.

21. The injectable aprepitant emulsion formulation of claim 1, wherein the initial pH of the composition is at least about 9.9.

22. A method of preparing an aprepitant emulsion, comprising:
a) providing an aqueous phase which includes water and at least one optional ingredient selected from the group consisting of tonicity agents, pH adjusting agents, bases, buffers, and pharmaceutically acceptable excipients;
b) providing an oil phase which includes aprepitant, a phosphatidylcholine emulsifier, a cosolvent, and an amount of a pH adjuster, e.g., sodium oleate or oleic acid, sufficient to provide the emulsion formed by combining the water and oil phases with an initial pH of at least 9.5, said oil phase optionally containing a surfactant;
c) combining the oil phase with the aqueous phase under conditions sufficient to form an oil in water emulsion, wherein the emulsion has an initial pH of at least 9.5; and
d) milling the emulsion, optionally with a tonicity agent;
whereby an aprepitant emulsion composition is formed.

23. A method of treating or preventing nausea in a patient, comprising administering an effective amount of injectable aprepitant emulsion of claim 1 to a patient in need thereof.
